(19) United States
(12) Patent Application Publication
(10) Pub. No.: US 2018/0160722 A1
(43) Pub. Date: Jun. 14, 2018

(54) VAPOR PRODUCTION SYSTEM

(71) Applicant: VMR Products LLC, Miami, FL (US)

(72) Inventors: Jan Andries VERLEUR, Miami Beach, FL (US); Dan RECIO, Miami Beach, FL (US); Zhiyuan LIU, Miami, FL (US); Hans VERLEUR, El Dorado, CA (US)

(73) Assignee: VMR Products LLC, Miami, FL (US)

(21) Appl. No.: 15/842,042

(22) Filed: Dec. 14, 2017

Related U.S. Application Data
(60) Provisional application No. 62/433,995, filed on Dec. 14, 2016.
(60) Provisional application No. 62/485,216, filed on Apr. 13, 2017.

(51) Int. Cl. A24B 15/16; A24F 47/00; F17C 7/04; H05B 1/02

(52) U.S. Cl. CPC A24B 15/167; A24F 47/008; F17C 2227/0393; H05B 1/0244; H05B 2203/021; F17C 7/04

(57) ABSTRACT

A device for producing inhalable vapor is disclosed. The device including an air pump, a heater, a supply of vapor produce, a compressor, and an outlet. The vaporizable material is heated to a vaporization temperature in the heater to produce vapor, which is mixed with a supply of fresh air from the air pump. The mixture is compressed in the compressor and delivered to a storage container through the outlet.

### CROSS REFERENCE TO RELATED APPLICATIONS

This application claims the benefit of U.S. Provisional Application Ser. No. 62/433,995, filed Dec. 14, 2016 and U.S. Provisional Application Ser. No. 62/485,216, filed Apr. 13, 2017, the disclosures of each of which are hereby incorporated herein by reference in their entireties.

### BACKGROUND

### 1. Technical Field Text

This disclosure relates generally to vaporizers, and more particularly to a vapor production system capable of storing vapor.

### 2. Background Information

Vaporizers have recently emerged as a new product for providing nicotine and other products through a smokeless inhalation process. There are many embodiments of vaporizers including the electronic cigarette. Most implementations consist of a power supply (typically a battery) and an atomizing device. In reusable electronic cigarettes the two items are separated into a battery and a cartomizer, to allow the disposal and replacement of the nicotine containing fluid cartomizer while preserving the more costly battery and associated circuitry (microcontroller, switch, indicating LED, etc.) In disposable electronic cigarettes the two items are combined to integrate the functions into one unit that is disposed of after either the battery energy or the nicotine containing E-liquid is exhausted. Vaporizers are generally designed to provide a supply of vapor on demand to a single user. In instances where multiple users are present, each user typically has their own vaporizer.

The E-liquid that is used to produce vapor in electronic cigarettes is generally a solution of one or more of propylene glycol (PG) and/or vegetable glycerin (VG) and/or polyethylene glycol 400 (PEG400) mixed with concentrated flavors, and optionally, a variable percentage of a liquid nicotine concentrate. This liquid may be termed an “E-liquid” and is often sold in a bottle or in disposable cartridges or cartomizers. Many different flavors of such E-liquids are sold, including flavors that resemble the taste of regular tobacco, menthol, vanilla, coffee, cola and various fruits. Various nicotine concentrations are also available, and nicotine-free E-Liquids are also common.

### BRIEF SUMMARY

In one aspect of the disclosure, a vapor production device is described. The vapor production device includes a supply of vaporizable material, a heater, an air pump, a compressor, and an outlet. The heater is in fluid communication with the supply of vaporizable material and is configured to heat the vaporizable material to a vaporization temperature. The air pump is in fluid communication with the heater and is configured to supply ambient air to the heater. The compressor is in fluid communication with the heater and is configured to compress a mixture of the ambient air and vapor produced in the heater. The outlet is configured to deliver the compressed mixture to a storage container.

Another aspect of the disclosure describes vapor production devices that comprise an air inlet, an air pump, a heater, a compressor and an outlet in fluid connection with each other; the air pump configured to supply ambient air to the heater; the heater configured to heat a vaporizable material to a vaporization temperature and to supply a vaporized material to the compressor; the compressor configured to compress a mixture of the ambient air and vapor produced in and supplied by the heater, and to deliver the mixture to the outlet; and the outlet configured to deliver the compressed mixture to a storage container.

In another aspect of the disclosure, a method of producing vapor is described. The method includes loading a vaporizable material into a vapor production device, heating the vaporizable material to a vaporization temperature to produce vapor, delivering ambient air for mixing with the vapor, mixing the ambient air with the vapor, compressing the air and vapor mixture, and delivering the compressed mixture to a storage container for storage of vapor mixture.

Yet another aspect of the disclosure describes methods for producing inhalable vapor, that comprise supplying a vaporizable material; loading the vaporizable material into the vapor production device as described herein; heating the vaporizable material to a vaporization temperature to produce vapor; delivering ambient air for mixing with the vapor; mixing the ambient air with the vapor; compressing the air and vapor mixture; and delivering the compressed mixture to the device outlet.

### BRIEF DESCRIPTION OF THE DRAWINGS

FIG. 1 illustrates a schematic view of a vapor compression system.

FIG. 2 illustrates a flowchart of a method of producing inhalable vapor.

FIG. 3 illustrates a storage container according to an aspect of the present disclosure.

### DETAILED DESCRIPTION

The following detailed description and the appended drawings describe and illustrate some embodiments of the disclosure for the purpose of enabling one of ordinary skill in the relevant art to make and use these embodiments. As such, the detailed description and illustration of these embodiments are purely illustrative in nature and are in no way intended to limit the scope of the disclosure in any manner. It should also be understood that the drawings are not necessarily to scale and in certain instances details may have been omitted, which are not necessary for an understanding of the embodiments, such as details of fabrication and assembly. In the accompanying drawings, like numerals represent like components.

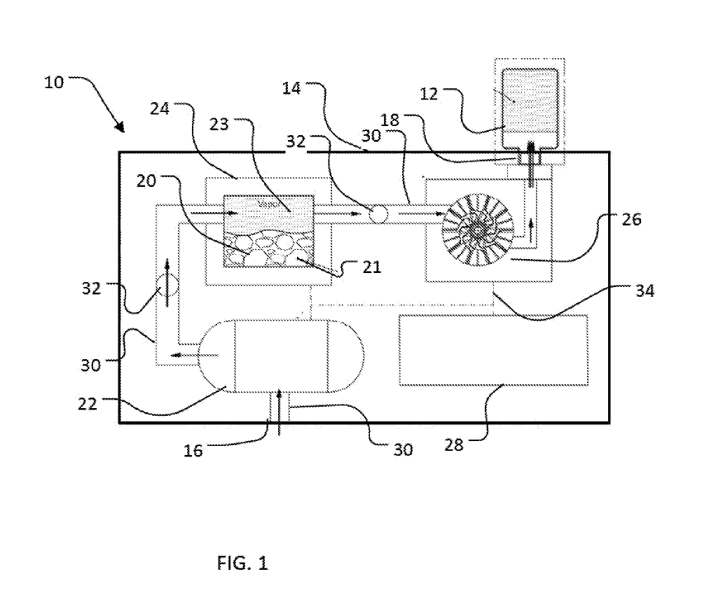

FIG. 1 illustrates a schematic view of a vapor production system 10. Vapor production system 10 is generally a system for producing compressed vapor suitable for filling a container 12 for later consumption by an end user. Once filled by vapor production system 10, container 12 may be used to consume vapor absent the vapor production system 10. Thus, vapor may be available in a setting that may not allow a traditional vaporizer, or in situations in which a high vapor production rate is required.

Vapor production system 10 is contained within a portable case 14. Portable case 14 is powered by way of batteries, an external power source, or a combination of batteries and an external power system. Portable case 14 has an outer surface with an inlet 16 for receiving air, an outlet 18 for produced vapor, and vaporizable material chamber 20 for receiving vaporizable material to be vaporized. The vaporizable material chamber 20 may receive cartridges containing particular types of vaporizable materials, or it may receive the raw vaporizable materials to be vaporized.

Contained within portable case 14 are components for producing vapor. Also contained within some embodiments of the vapor production system or its portable case are components for compressing vapor. In yet other embodiments, components for both producing vapor and for its compression are contained within the vapor production system or its portable case. In the embodiment of FIG. 1, the components includes an air pump 22, a heating center 24, a vapor compressor 26, and a control panel 28. Additionally, each component is in fluid communication with adjacent components though air channels 30 with fluid flow between components controlled by valves 32. Control panel 28 is wired to each component and one or more valves 32 through common techniques as known by one of ordinary skill in the art. Dashed line 34 indicates the connection between control panel 28 and the components.

Air pump 22 receives ambient air through inlet 16. Inlet 16 may be connected directly to air pump 22, or as shown in the embodiment of FIG. 1, inlet 16 may connect to air pump 22 by way of air passage 32. Inlet 16 may have a filter suitable to prevent foreign material from entering air pump 22. Air pump 22 may be any type of air pump as known in the art. Air pump 22 directs a volume of air thorough an air channel 30 to heating center 24. A first valve 32 is present in air channel 30 and prevents air from flowing into heating center 24 from air pump 22 when not in use. Valve 32 may be adjustable, to regulate the flow of air, or act as a simple on/off valve.

Air channel 30 is in fluid communication with heating center 24. Heating center 24 has a chamber 20 for receiving vaporizable material 21 and a heater for vaporizing vaporizable material 21. Heating center 24 may heat vaporizable material 21 to produce vapor 23 as is known in the art. Air supplied through air channel 30 mixes with vapor 23 to produce an inhalable vapor.

A second air channel 30 directs inhalable vapor from heating center 30 into vapor compressor 26. Vapor compressor 26 receives inhalable vapor, compresses it, and directs the compressed vapor to outlet 18. Vapor container 12 is secured to outlet 18 to receive the compressed vapor. Vapor container 12 may be attached, connected or secured to portable case 14 or vapor production system 10 during filling using common techniques such as a threaded connection or a twist lock connection. The compressed vapor contained within the vapor container 12 may then be used independently of the vapor production device 10. Additionally, the compressed vapor may be delivered at a much higher flow rate than what is available using a standard vaporizer. Because the vapor is compressed, vapor container 12 may hold a larger quantity than would otherwise be possible.

Control panel 28 is configured to control the components of the vapor control system. Control panel 28 regulates the power delivered to the heating center 24 to convert the vaporizable material to vapor at a temperature suitable for its vaporization. The suitable temperature may be entered manually by an operator, or control panel 28 may have stored data indicating an optimal temperature or heating profile. The quantity of air delivered to heating center 24 may be or is controlled by the control panel 28 and may be adjusted in combination with the power delivered to the heating center for optimal vapor production. Compressor 26 is further controlled by control panel 28 to deliver an optimal pressure for storage of the inhalable vapor. Depending on the type of vapor container and the vaporizable material being vaporized, compressor 26 may regulate the level of compression to which the inhalable vapor is compressed.

Once vapor container 12 is filled with vapor mixture to a desired pressure, vapor container 12 may be detached or otherwise disconnected from portable case 14 or vapor production system 10.

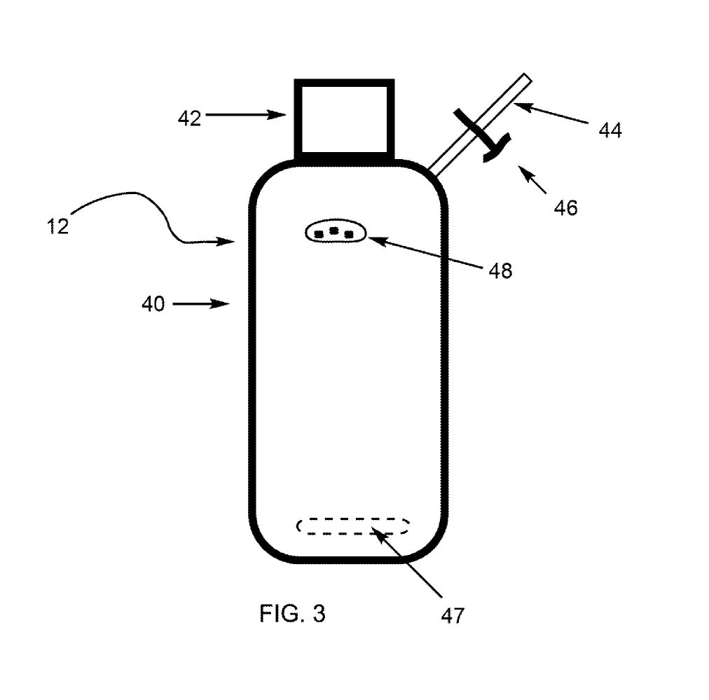

Vapor container 12 may be a small can shaped container having a neck for interfacing with the vapor production system 10, for example as illustrated in FIG. 3. Materials of construction or design of the receiving container are not critical so long as the container can safely withstand the pressure level of vapor output from system 10. Vapor container 12 is refillable and reusable. The container typically has a body 40 of sufficient durability and/or design to withstand pressures introduced during delivery of the compressed vapor. The container also has a connector portion 42 configured to be compatible with outlet 18. A vapor delivery device such as an inhalation tube or pipe may be connected to the neck of the vapor container 12. In other embodiments, vapor container 12 may have a separate connection or tube 44 for delivering pre-produced inhalation vapor. In certain other embodiments, the connection or tube for delivering pre-produced vapor may include a valve 46 or other regulating device to assist in delivery of vapor or to depressurize the container. In some embodiments, the interior of the vapor container 12 may be accessible for cleaning between fillings of different vaporizable materials. Alternatively, in other embodiments, the vapor container 12 may be sealed.

To prevent the vapor from condensing, or to reestablish the vapor properties within the container prior to inhalation, may require an establishing or maintenance of vapor temperature. To facilitate heat retention, the vapor container 12 may be insulated to inhibit the loss of heat from the vapor container 12, maintaining its temperature without external heating. Alternatively, vapor container 12 may be actively heated to prevent vapor from condensing. The vapor container 12 may have its own controller 48 including an integrated heater 47 for controlling the temperature of the vapor. Typically the heater is configured to maintain a pressurized vapor in a vapor state.

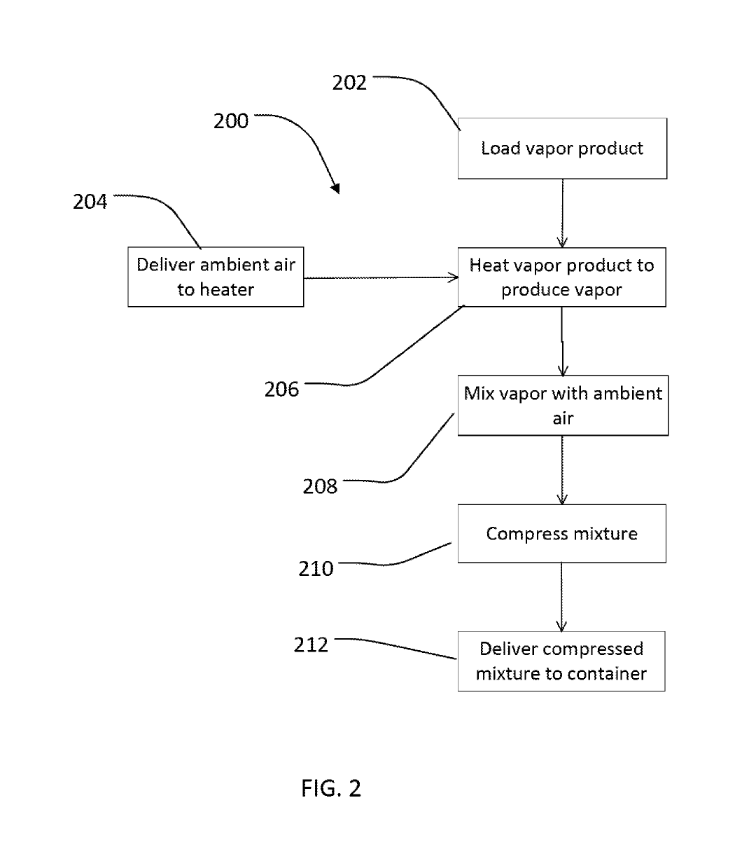

FIG. 2 illustrates a method 200 of producing inhalable vapor. In block 202, a vapor production device, such as vapor production device 10 is loaded with a vapor vaporizable material, such as E-liquid. At block 204, ambient air is delivered to a heater, such as heating center 24. The ambient air may be delivered to the heater by way of air pump 22. At block 206, the vaporizable material is heated to vaporize the material. Heating center 24 may heat the vaporizable material to produce the vapor. The vapor is mixed with the supply of ambient air at block 208 and compressed at block 210. Compressor 26 may be used to compress the mixture of ambient air and vapor. The compressed mixture of ambient air and vapor is then delivered to a suitable container at block 212, such as vapor container 12.

Some embodiments of the present disclosure are directed to vapor production device kits comprising a vapor production device as described herein and instructions for operating the vapor production device. In some embodiments, the kit further comprises a container as described herein capable of storing pressurized vapor produced by the device.

The descriptions set forth above are meant to be illustrative and not limiting. Various modifications of the invention, in addition to those described herein, will be apparent to those skilled in the art from the foregoing description. Such modifications are also intended to fall within the scope of the concepts described herein. The disclosures of each patent, patent application and publication cited or described in this document are hereby incorporated herein by reference, in their entireties.

The foregoing description of possible implementations consistent with the present disclosure does not represent a comprehensive list of all such implementations or all variations of the implementations described. The description of some implementation should not be construed as an intent to exclude other implementations. For example, artisans will understand how to implement the invention in many other ways, using equivalents and alternatives that do not depart from the scope of the invention. Moreover, unless indicated to the contrary in the preceding description, none of the components described in the implementations are essential to the invention. It is thus intended that the embodiments disclosed in the specification be considered as illustrative, with a true scope and spirit of the invention being indicated by the following claims.

1. A vapor production device, comprising:
an air inlet, an air pump, a heater, a compressor, and an outlet; in fluid connection with each other;
the air pump configured to supply ambient air to the heater;
the heater configured to heat a vaporizable material to a vaporization temperature and to supply a vaporized material to the compressor;
the compressor configured to compress a mixture of the ambient air and vapor produced in and supplied by the heater, and to deliver the mixture to the outlet; and
the outlet configured to deliver the compressed mixture to a storage container.

2. The vapor production device according to claim 1, further comprising a supply of vaporizable material.

3. The vapor production device according to claim 1, further comprising a storage container suitable for storage of pressurized vapor produced by the vapor production device.

4. The vapor production device according to claim 1, wherein the storage container is insulated.

5. The vapor production device according to claim 1, wherein the storage container includes a heater configured to maintain a pressurized vapor in a vapor state.

6. A method for producing inhalable vapor, comprising:
supplying a vaporizable material;
loading the vaporizable material into the vapor production device according to claim 1;
heating the vaporizable material to a vaporization temperature to produce vapor;
delivering ambient air for mixing with the vapor;
mixing the ambient air with the vapor;
compressing the air and vapor mixture; and
delivering the compressed mixture to the device outlet.

7. The method for producing inhalable vapor according to claim 6, further comprising:
delivering the compressed mixture to a storage container for storage of a compressed vapor mixture.

8. A kit comprising a vapor production device according to claim 1 and instructions for operating the vaporizer.

9. The kit according to claim 8, further comprising a storage container suitable for storage of pressurized vapor produced by the vapor production device.

10. The kit according to claim 9, wherein the storage container is insulated.

11. The kit according to claim 8, wherein the storage container includes a heater configured to maintain a contained pressurized vapor in a vapor state.
